(19) United States
(12) Patent Application Publication
(10) Pub. No.: US 2025/0001701 A1
(43) Pub. Date: Jan. 2, 2025

(54) 3D Bioprinters

(71) Applicant: Cellink Bioprinting AB, Göteborg (SE)

(72) Inventors: Hector Martinez, Göteborg (SE); Erik Gatenholm, Göteborg (SE)

(73) Assignee: Cellink Bioprinting AB, Göteborg (SE)

(21) Appl. No.: 18/882,420

(22) Filed: Sep. 11, 2024

Related U.S. Application Data
(63) Continuation of application No. 16/962,668, filed on Jul. 16, 2020.
(60) Provisional application No. 62/621,091, filed on Jan. 24, 2018.

(30) Foreign Application Priority Data
Jan. 24, 2018 (SE) 1850073-6

(51) Int. Cl. B29C 64/393; B29C 64/227; B29C 64/245; B33Y 10/00; B33Y 30/00; B33Y 50/02; C12M 1/12; C12M 1/26

(52) U.S. Cl. CPC B29C 64/393; B29C 64/227; B29C 64/245; C12M 25/14; C12M 33/00; B33Y 10/00; B33Y 30/00; B33Y 50/02

(57) ABSTRACT

The present disclosure relates to a bioprinter system comprising a printbed, at least one of exchangeable and/or fixed toolhead, a cell culture monitor device, the device comprising a camera arranged to provide images of a cell culture or construct at the printbed, and a processing element arranged to monitor cell status at the printbed based on the provided images.

### TECHNICAL FIELD

The present disclosure relates to a 3D bioprinter.

### BACKGROUND

Bioprinting, an additive manufacturing technology, has gained due attention for its ability to spatially control the placement of cells, biomaterials and biological molecules. Consequently, it offers endless possibilities to the future of tissue and organ regeneration, basic research and drug screening.

The 3D bioprinter is able to dispense materials while moving in X, Y, and Z directions. This enables the engineering of complex structures from the bottom up. Moreover, this technology allows the biofabrication of biomimetic-shaped 3D structures unique to the target tissue or organ, since it can be combined with CAD/CAM technology using patients' medical images. In a pre-bioprinting process, a model for use by the 3D bioprinter is created. Further, materials that will be used are chosen. Common technologies used for bioprinting are computed tomography (CT) and magnetic resonance imaging (MRI). To print with a layer-by-layer approach, tomographic reconstruction can be done on the images. The now-2D images can then be used by the printer.

The inventors of the present invention has identified a need for an improved bioprinter system, which provides for improved results in bioprinting.

### SUMMARY

In accordance with the present invention a bioprinter system comprises a printbed, at least one of an exchangeable and/or fixed toolhead and a cell culture monitor device. The monitor device comprises a camera arranged to provide images of a cell culture or construct at the printbed. The system further comprises a processing element arranged to monitor cell status in the cell culture or construct at the printbed based on the provided images.

Further, a method is disclosed for monitoring a cell culture or construct at a bioprinter printbed. The method comprises providing images of the cell culture or construct, and monitoring cell status at the printbed based on the provided images.

### SHORT DESCRIPTION OF THE DRAWINGS

FIG. 1 shows a bioprinter system.

FIG. 2 illustrates one aspect of the present disclosure.

FIG. 3 illustrates other aspects of the present disclosure.

FIG. 4 illustrates further aspects of the present disclosure.

### DETAILED DESCRIPTION

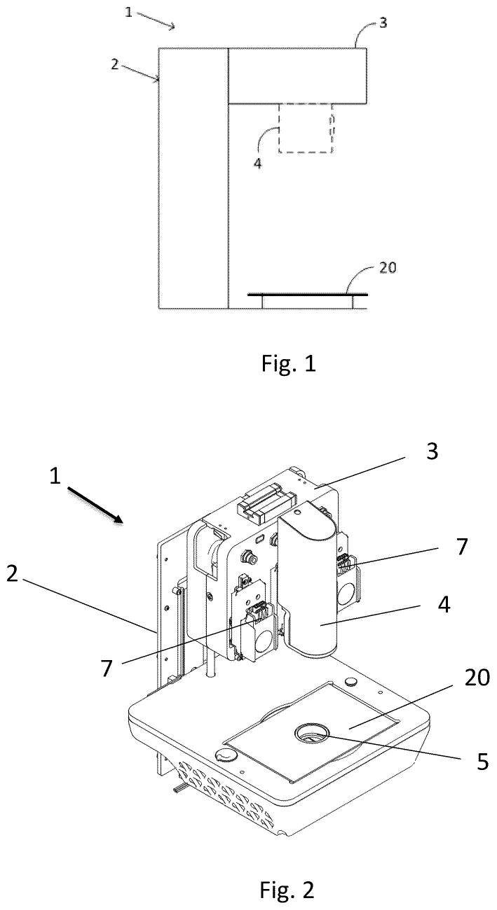

FIG. 1 discloses a schematic view of an example of a 3D bioprinter 1 as disclosed herein. The 3D bioprinter can be used for manufacture of three-dimensional engineered biological tissues. The 3D bioprinter 1 may be used in printing constructs that are suitable for use in any of the applications chosen from: implants in the animal or human body, such as repairing or replacing tissue, topical applications, cosmetic applications and drug test applications, as well as any other suitable applications. Printed constructs may include, but are not limited to, tissue-like constructs, constructs with or without living cells, cell cultures, tissue implants and three-dimensional matrices.

The 3D bioprinter 1 may comprise a base unit 2. The base unit 2 may comprise a support 3 adapted for mounting of at least one toolhead 4. In some aspects the support 3 is adapted to accommodate a plurality of toolheads 4.

FIGS. 2, 3 and 4 illustrate a number of different aspects of a 3D bioprinter as disclosed herein. Notably, even though specific combinations are shown in the figures and described herein, it is understood that specific features may be combined in further suitable manners.

In one aspect, a support 3 is adapted for mounting of at least one exchangeable toolhead 4. Thus, as seen in FIGS. 2-4, the support may comprise one or more attachment elements 7, adapted for mounting a toolhead 4. In the figures, one single toolhead 4 is shown as mounted on the support 3, and it is understood that such toolhead may be either integrated or exchangeable. Further, the term “integrated” may mean either permanently integrated or mounted in a non-exchangeable manner.

If one or more exchangeable toolhead(s) are used, such toolheads may then be dismounted and displaced with other toolhead(s). In one example, the support 3 is adapted for mounting of at least one non-exchangeable toolhead. In another example, the support may be adapted for mounting of at least one exchangeable toolhead 4 and at least one non-exchangeable toolhead 4 (not illustrated).

In the example where the support 3 is adapted for mounting of at least one exchangeable toolhead, the 3D bioprinter becomes very flexible and efficiency in operation of the 3D bioprinter can be increased. For example, the user can prepare the toolhead outside the 3D bioprinter, wherein the preparation may for example involve loading material and/or selecting needle, and/or calibrating or setting up a sensor or other device. Thereafter the currently used toolhead can be substituted with the new one.

Thereby the bioprinter does not need to be stopped for time consuming activities such as loading of material and/or changing needle, calibration or setting up. Instead, a short stop can be made to substitute the currently used toolhead with the prepared one. Alternatively, the currently used toolhead can be exchanged when printing. For example, if the support 3 is adapted to mount a plurality of toolheads, the exchange can easily be done while printing.

The toolhead can be inserted or removed by a user with or without use of a tool. Alternatively, the toolhead can be automatically inserted and/or removed by the 3D bioprinter itself.

The connection of a toolhead can require one or a plurality of steps. It may for example involve inserting a nut and/or connecting a cable and/or connecting gas supply.

The at least one toolhead 4 may include at least one actuating toolhead and/or at least one sensor toolhead. An actuating toolhead may also be referred to as a printhead, especially in those cases where it adapted for bioprinting. Thus, the expression “toolhead/is meant to encompass all types of tools used in a bioprinter. Examples of actuating toolheads include pneumatic extrusion toolheads, syringe pump toolheads, inkjet toolheads, high temperature extrusion toolheads, toolheads for removal of material and/or curing toolheads. Toolheads for removal of material may comprise a knife and/or a laser toolhead and/or a milling toolhead and/or a drilling toolhead and/or a needle and/or tip adapted for suction and/or addition of material. The curing toolheads may comprise a curing UV toolhead and/or a visible light curing toolhead and/or a laser light curing toolhead. Other examples of actuating toolheads include exhaust gas toolheads for exhaust of gas such as air.

The actuating toolhead may be provided with sensor(s). Examples of sensing toolheads include camera toolheads, probing toolheads and/or 3D scanning toolheads.

A bioprinter system according to the present disclosure may be arranged in several different manners. A number of different combinations are disclosed herein, all comprising a cell culture monitor device within or arranged in connection to a bioprinter. The cell culture monitor may be any type of monitor capable of monitoring cell status, such as cell growth, cell viability, histology, cell distribution, cell density, number of cells and/or types of cells. Further, the term “cell culture” in intended to encompass all types of environments wherein living cells may be sustained and cultured. Examples include, but are not limited to, standard cell culture vessels such as petri dishes, multi-well culture vessels, microscope slides, cell culture flasks and tubes etc., but also within a bioprinter construct, such as within a matrix of supporting material. The monitor device may further be adapted to sense, map or detect e.g. cell or tissue markers, such as for instance fluorescently tagged markers, enzymatically tagged markers and/or immunologically tagged markers.

A cell culture monitor device as disclosed is preferably intended for monitoring of the cells in a bioprinted construct or cell culture, during the bioprinting process, and/or after bioprinting in the bioprinter system. Further, it is also possible that the cell culture monitor device may be used to monitor cells in a construct, cell culture or tissue to be used in the bioprinter before and/or during bioprinting.

The cell culture monitor may be fully or partially automated in its function. Further, the cell culture monitor may comprise a cell counter.

A cell culture monitor 5 may comprise a camera of any suitable type, such as a standard photography camera, a video camera, an infrared camera, a camera adapted to detect specific ranges of light, a microscope or other imaging device. The term “camera” is herein intended to comprise, be part of or be any type of optical imaging system capable of monitoring cell status by obtaining images. Such images may be live, still or video images.

In one aspect, as schematically illustrated in FIG. 2, a bioprinter system 1 comprises a cell culture monitor device 5 that fits in the printbed 20 of a bioprinter. The cell culture monitor device 5 is in this example an exchangeable device arranged to be received by the printbed 20 or mounted in connection to the printbed 20. It may be provided in a dedicated mounting space, such as a hole or recess in the printbed 20, preferably such that the device is essentially flush with the top surface of the printbed when arranged in the printbed. As an alternative, not illustrated, the cell culture monitor device 5 may be provided on top of the printbed 20, preferably having a flat and wide enough top surface for a petri dish or other type of cell culture container to be placed on top of the monitor device 5.

Once detached from the bioprinter, the cell culture monitor device 5 can preferably also operate inside any standard cell culture incubator to allow the user to monitor the cells and evaluate the cells optimum culture conditions. The cell culture monitor device 5 comprises a camera arranged to provide live, video or still images of a cell culture or construct comprising cells, when placed on top of, or above, the monitor device 5. In one aspect, the camera may provide direct live images. In another aspect, the camera may provide still images. These images may be provided in response to a command by a user, or at one or several specified time points, such that the cell culture may be monitored over time. In a third aspect, the camera may provide video images.

Depending on the type of camera used, as well as the ambient light provided around the printbed, an appropriate light source 6 may be provided at a suitable location, such as on the toolhead 4 or support 3, such that light is supplied to the location where the cell culture or construct is arranged (not shown). Such a light source may further be arranged as an attachment to the monitor device 5 itself (not shown), such that when the monitor device 5 is detached from the bioprinter, the light source will remain connected to the device 5 and continue to illuminate the cell culture.

The bioprinter comprises a processing element arranged to monitor cell status at the printbed 20. Cell status may any parameter used to monitor cells in culture or tissue, such as, but not limited to one of or any combination of: cell growth, cell count, cell viability, cell shape, cell size, cell differentiation, cell density, cell distribution, confluency of cells, cell type or infection by other cell types. The processing element uses information acquired from the obtained images to monitor cell status. It may also use information based on the spatial relation between the camera and the monitored cell culture or construct.

Further, the monitor device 5 may be integrated with analysis software adapted to monitor the cells over a prolonged and continuous period of time, preferably automatically. For example, the monitor device 5 and analysis software may be arranged to obtain time lapse videos of the cell status. For instance, the system could also analyze the confluency over the cell culture period and the processing element may preferably provide warnings to the user, for example by sending a text message and/or email, when something goes wrong with cell culture status or if there is an indication of contamination or infection in the cell culture. In other words, the bioprinter system may be arranged to provide an alert signal when the cell culture status reaches at least one predetermined criteria.

The bioprinter system may be adapted such that the camera is arranged to perform a scanning movement to scan the cell culture or construct at the printbed 20. In addition, a bioprinter system with a cell culture monitor device 5 may be designed to scan and analyze wellplates as well as any cell culture tissue flask, vessel and surface, such as a microfluidic device. Such a system preferably moves the camera instead of moving the samples or construct. This makes the system very fast and accurate in scanning and analysis. A bioprinter system with a camera arranged for scanning and movement preferably comprises a control element arranged to obtain control signals for controlling the scanning movement. The scanning movement of the camera may be controlled manually by a user and/or automatically by the system.

The scanning movement is preferably performed by an actuator, and/or motor, and controlled by the control element.

The system could also include an automated cell counter by including any disposable and/or reusable hemocytometer. In such a setup, the system could perform a single or multiple measurements to evaluate important parameters such as cell viability, concentration, shape, size, among others. The automatic cell counter feature allows a user to evaluate the cell numbers and viability before a bioprinting experiment. These two factors are extremely important to control and evaluate before embedding the cells in the bioink, in order to guarantee a desired final cell concentration in the printed tissue. The system may also provide valuable information of cell status during and after a bioprinting process.

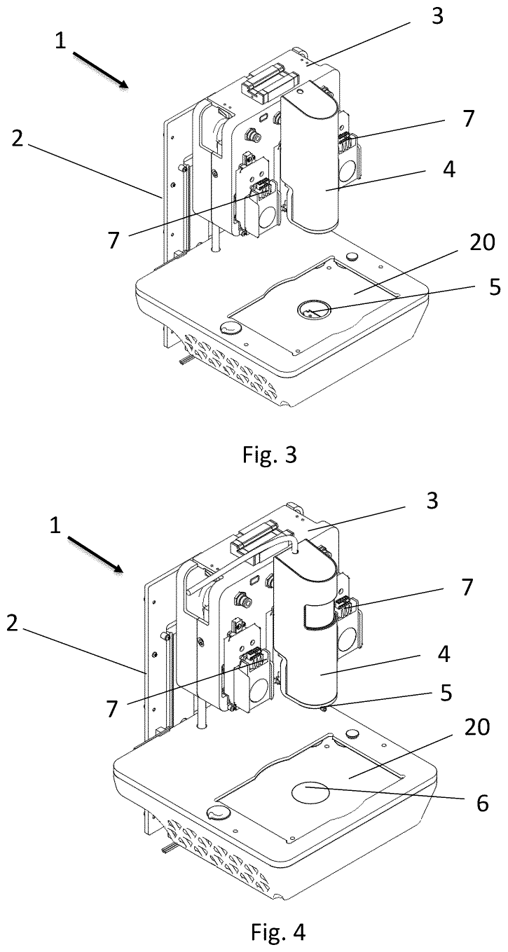

In another aspect, illustrated in FIG. 3, the cell culture monitor device 5 may be integrated in the printbed of a bioprinter. It may be located anywhere in the printbed; center, side, back, front, as well as on the side of the printbed, e.g. as a plugin device. It may be arranged under, on top of or in the printbed, preferably essentially flush with the top surface of the printbed. The user may use this device to monitor the cells over the cell culture period and evaluate their status, such as cell growth, cell count or cell viability.

All other aspects of the bioprinter 1 and cell culture monitor device 5 shown in FIG. 3 may be as described above in connection with FIG. 2. Thus, the cell culture monitor device 5 comprises a camera arranged to provide live, video or still images of a cell culture, or construct comprising cells, arranged on the printbed 20 and monitor device 5.

Further, the monitor device 5 may be integrated with analysis software to automatically monitor the cells over a prolonged and continuous period of time. In addition, the device may be adapted to scan and analyze different types of cell culture surfaces, and the device may be adapted to move the camera instead of moving the cell culture when scanning.

The device may be adapted to be used as a cell counter. The automatic cell counter feature allows a user to evaluate the cell numbers and viability before a bioprinting experiment. These two factors are extremely important to control and evaluate before embedding the cells in the bioink in order to guarantee a desired final cell concentration in the printed tissue.

In a third aspect, as illustrated in FIG. 4, the bioprinter system 1 may comprise a cell culture monitor device 5 that is mounted above the printbed. Such a monitor device 5 may be e.g. integrated in, part of, or attached to an exchangeable or fixed toolhead 4 of a bioprinter.

In the case of a monitor device 5 being part of or comprised by an exchangeable toolhead 4, once detached from the bioprinter, such a cell culture monitor device 5 may be adapted to be mounted on a stand-alone mount or holder to be able to operate inside any standard cell culture incubator, for instance to allow the user to monitor cells and evaluate the cells optimum culture conditions. The monitor device 5 may also be adapted to be used without a holder inside any standard cell culture incubator.

In one aspect, the bioprinter may comprise an appropriate light source 6 at a suitable location, such as in or near the printbed 20, such that light is supplied to the location where the cell culture or construct is arranged. In the example of FIG. 4, a light source 6 is mounted within the printbed 20. Such a light source may further be arranged as an attachment to the monitor device 5 itself (not shown), such that when the monitor device 5 is detached from the bioprinter, the light source will remain connected to the device 5 and continue to illuminate the cell culture or construct.

All other aspects of a bioprinter and cell culture monitor device 5 as shown in FIG. 4 may be as described above in connection with FIGS. 2 and 3. Thus, the cell culture monitor device 5 comprises a camera arranged to provide live, video or still images of a cell culture, or construct comprising cells, arranged on the printbed 20.

Further, the monitor device 5 may be integrated with analysis software to automatically monitor the cells over a prolonged and continuous period of time. Further, the device may be adapted to scan and analyze different types of cell culture surfaces.

In the bioprinter shown in FIG. 4, since the monitor device 5 may be attached to or integrated with a toolhead or toolhead holder, it may further be adapted to move the camera, for instance in in X and Y axes, instead of moving the cell culture or construct. This makes the system very fast and accurate in scanning and analysis.

The monitor device 5 may be adapted to be used as a cell counter. The automatic cell counter feature allows a user to evaluate the cell numbers and viability before bioprinting experiments. These two factors are extremely important to control and evaluate before embedding the cells in the bioink in order to guarantee a desired final cell concentration in the printed tissue.

The present invention is not limited to the above-described preferred embodiments. Various alternatives, modifications and equivalents may be used. Therefore, the above embodiments should not be taken as limiting the scope of the invention, which is defined by the appending claims.

1. A bioprinter system comprising
a printbed,
at least one of an exchangeable and/or fixed toolhead,
a cell culture monitor device, the device comprising a camera arranged to provide images of a cell culture or construct at the printbed, and
a processing element arranged to monitor cell status in the cell culture or construct at the printbed based on the provided images,
wherein the cell culture monitor device is an exchangeable device.

2. The bioprinter system according to claim 1, further comprising a light source arranged to illuminate the cell culture or construct at the printbed.

3. The bioprinter system according to claim 1, wherein the camera provides still images.

4. The bioprinter system according to claim 1, wherein the camera provides video images.

5. The bioprinter system according to claim 1, wherein the camera provides images at specified time points.

6. The bioprinter system according to claim 1, further comprising a hemocytometer arranged to perform a cell count in the cell culture or construct.

7. The bioprinter system according to claim 1, wherein the processing element is arranged to determine a cell viability and/or concentration based on the acquired images and/or cell count.

8. The bioprinter system according to claim 1, wherein the processing element is arranged to determine a cell size and/or cell shape of the cell culture or construct based on the acquired images and/or the cell count.

9. The bioprinter system according to claim 1, wherein the processing element is arranged to analyze a confluency over a cell culture period based on the acquired images and/or the cell count.

10. The bioprinter system according to claim 1, wherein the processing element is arranged to provide an alert signal when the cell culture status reaches at least one predetermined criteria.

11. The bioprinter system according to claim 10, further comprising a communication element arranged to provide a warning to a user upon receipt of the alert signal, wherein the warning is a text message, an e-mail message and/or an audio signal and/or a visual signal or display.

12. The bioprinter system according to claim 1, wherein the camera is arranged to perform a scanning movement to scan the cell culture or construct at the printbed.

13. The bioprinter system according to claim 12, further comprising a control element arranged to obtain control signals for control of the scanning movement.

14. The bioprinter system according to claim 13, further comprising an actuator or motor arranged to perform the scanning movement, said actuator or motor being controlled by the control element.

15. The bioprinter system according to claim 12, wherein the processing element is arranged to monitor cell status at the printbed based also on information related to a spatial relation between the camera and the cell culture or construct, based on a control signal.

16. The bioprinter system according to claim 1, wherein the monitor device is arranged to be received by or being comprised in the printbed.

17. The bioprinter system (1) according to claim 1, wherein the monitor device is integrated in, part of, or attached to an exchangeable or fixed toolhead.

18. The bioprinter system according to claim 1, wherein the exchangeable device when detached from the bioprinter can also operate inside any cell culture incubator for monitoring the cells in the cell culture or construct.

19. The bioprinter system according to claim 1, wherein the monitor device is formed by the at least one exchangeable bioprinter toolhead.

20. The bioprinter system according to claim 1, wherein the monitor device is arranged to be received by the printbed, and is provided in a dedicated mounting space, said mounting space being either a hole or recess in the printbed.

21. The bioprinter system according to claim 20 wherein the dedicated mounting space and the monitor device are configured such that the monitor device is essentially flush with the top surface of the printbed when arranged in the printbed.

22. The bioprinter system according to claim 1, wherein the monitor device is arranged to be mounted in connection to the printbed, such that the monitor device is arranged on top of the printbed when mounted.

23. The bioprinter system according to claim 22, wherein a top surface of the monitor device has a flat surface adapted to support a cell culture dish.

24. The bioprinter system according to claim 1, wherein the monitor device is mounted above the printbed.

25. The bioprinter system according to claim 1, wherein the monitor device is integrated in, part of, or attached to an exchangeable toolhead, and said toolhead, when detached from the bioprinter, is adapted to be mounted on a stand-alone mount or holder to be able to operate inside any standard cell culture incubator.
